(19) United States
(12) Patent Application Publication
(10) Pub. No.: US 2011/0065806 A1
(43) Pub. Date: Mar. 17, 2011

(54) EMOLLIENT COMPOSITION FOR THE PREVENTIVE TREATMENT OF ATOPIC DERMATITIS

(75) Inventors: Didier Coustou, Toulouse (FR); Hélène Duplan, Auzeville Tolosane (FR)

(73) Assignee: PIERRE FABRE DERMO-COSMETIQUE, Boulogne-Billancourt (FR)

(21) Appl. No.: 12/992,666

(22) PCT Filed: May 18, 2009
(86) PCT No.: PCT/EP2009/056007

§ 371 Date: Nov. 15, 2010

(30) Foreign Application Priority Data
May 16, 2008 (EP) 0853185

(51) Int. Cl. A61K 31/047; A61P 17/00

(52) U.S. Cl. 514/738

(57) ABSTRACT

The invention relates to a composition for topical use that comprises a combination of glycerol, Vaseline and liquid paraffin as an active principle, in the form of an oil-in-water or water-in-oil emulsion, said composition being used in the prevention of atopic dermatitis.

The present invention relates to the field of atopy and of the treatment of affections which are associated with it.

Atopy is defined as a hereditary predisposition to react in a symptomatic way (disease) to various allergens, such as house dust, mites, pollens, animal hairs, etc. Affections reputed to be atopic comprise atopic dermatitis (AD), allergic bronchial asthma and allergic rhinitis or “hay fever”.

Atopic dermatitis (or atopic eczema) is a frequent dermatological disease, affecting 10-15% of infants in France, with an increase over the last decades.

Atopic dermatitis is a chronic disease which develops with eruptions, interrupted with remission periods during which the lesions are always present but minimal, essentially expressed by xerosis (skin dryness) and pruritus.

Inflammation of the skin is characteristic of the eruptions which are expressed by inflammatory signs and symptoms comprising red patches (erythema), vesicles, scabs, exudation and itches.

The disease generally begins between the age of 3 months and of 2 years but may begin even earlier from 1 month of age. Its development is often favorable with complete remission occurring between 5 and 8 years of age in the majority of the cases. Resurgence at adolescence or in young adults is possible. The disease may persist in adulthood in 15-20% of the cases.

Development towards another atopic affection is also possible. The presence of AD during childhood increases the risk of developing asthma and there exists a clear correlation between the frequency of AD and the frequency of asthma. The risk of occurrence of asthma in a child affected with AD is estimated to be between 30 and 40%.

The risk of occurrence of a food allergy, most often before 3 years of age, or of allergic rhinitis, occurring later on, varies according to the studies.

As regards atopic dermatitis, there exists a marked genetic predisposition with a 30-50% risk of developing the disease if one of the parents is affected and a risk of close to 80% if both parents are affected. The genetic predisposition involves many genes, either coding for determinants of the barrier function of the epidermis, or for innate or adaptive immunity actors. Taking into account many recent investigations, the assumption may be issued that AD (or even other atopic diseases) are related to a primitive abnormality of the barrier function of the epidermis, responsible for increased permeability to environmental agents (allergens and microorganisms), responsible for immunologic activation and allergic symptoms.

The abnormality of the epidermal permeability is expressed by skin xerosis and by an increase in imperceptible water loss and by demonstration of the polymorphism of genes coding for epidermal proteins such as filaggrin (FLG), participating in the barrier function of the epidermis (Palmer, 2006). A recent study in France confirms the association of polymorphism of the FLG gene with AD, with a relative risk above 3 (Hubiche, 2007).

The atopy may be considered as a retarded hypersensitivy reaction to the contact with allergens of the environment. It generally appears in two phases. A first clinically silent sensitization phase generates T lymphocytes specific to the skin. This sensitization occurs by penetration of the allergens from the environment at the skin facilitated by xerosis. A second phase of activation of specific T lymphocytes results in the production of pro-inflammatory cytokines.

Globally, the outward signs of atopic dermatitis result from complex interactions between environmental factors, pharmacological abnormalities, dysfunctions or alterations of the barrier functions of the skin, immunological phenomena and genetic susceptibility.

Among the numerous factors, skin hyperreactivity, unsuitable immune response to various microorganisms as well as secondary infections seem to play a role in chronicity of the pathology.

The role of skin flora in the occurrence and/or the maintaining of immune activation favoring development of eczema lesions is increasingly better known. The alteration of the barrier function of the skin associated with some immunodeficiency in patients affected with atopic dermatitis causes colonization by Staphylococcus aureus which colonizes the skin of 90% of the patients having AD, even outside the progressive eruptions of the disease. (Current Opinion in Allergy and Clinical Immunol. 2007, 7, 413).

There exists a correlation between the seriousness of the disease and the staphylococcus colonization density on the one hand, and the positivity frequency of specific IgEs directed against staphylococcal toxins on the other hand (J. Allergy Clin. Immunol. 2006, 117, 1141; Am. J. Clin. Dermatol., 2006, 7, 273; Clin. Rev. Allergy Immunol., 2007, 33, 167; Current Opinion Allergy and Clinical Immunol. 2004, 4, 373).

These facts give rise to suspecting that the golden staphylococcus has a role in the development of local immune activation responsible for outward cutaneous signs of atopic dermatitis and perhaps of allergic sensitizations.

In addition to scratching, there are many factors promoting colonization by Staphylococcus aureus via alteration of the primary skin defense system and firstly the alteration of the lipid composition of the stratum corneum. This alteration of the barrier function of the skin is thus responsible for colonization by the staphylococci and also for an increase in the water loss at the skin, causing xerosis, irritation, itches, thereby creating a sort of physiopathological vicious circle.

Therapeutic management of atopic dermatitis may be considered at various levels, essentially symptomatic levels but also preventive levels. Today there is no curative treatment of the disease.

In addition to eviction of the identified allergens which is desirable as far as possible, moisturization of the skin as a means for restoring or maintaining its role of a barrier comes as an addition to the symptomatic treatment of eruptions by local corticotherapy which is the standard therapy of this disease. Other topic non-steroid anti-inflammatory agents (notably inhibitors of calcineurin) may also be used for treating the eruptions. Finally phototherapy, immunosuppressors per os or other extensive systemic therapies are reserved to severe forms.

However, the use of corticoids, of anti-inflammatory agents and/or immunosuppressors, even via a topic route, in particular in infants or toddlers, is not trivial, exposes to undesirable effects and is only a symptomatic treatment.

On the other hand, phobia exists towards the use of corticoids which does not facilitate the management of this disease nor compliance of the patients, which is often poor.

Finally, Staphylococcus aureus may induce resistance to corticoids (Clin. Rev. Allergy Immunol. 2007 33 167).

There thus exists a need and strong demand for therapeutic alternatives and more particularly for preventive treatments. These treatments aim at reducing the frequency and/or the intensity of the eczema eruptions which are observed in patients affected with atopic dermatitis. This approach is a tertiary preventive act according to the WHO definition since it tends to avoid complications (inflammatory eruptions) of a disease (atopic dermatitis) already present.

The application WO2008048076 discloses the use of glucosamine or one of its derivatives for treating atopic dermatitis.

Application WO2007023226 discloses the use of a probiotic combined with a prebiotic for treating atopic dermatitis in children.

Now, it has been noticed quite surprisingly and unexpectedly that a combination of the glycerol, vaseline® and liquid paraffin type in the form of an oil-in-water or water-in-oil emulsion allowed prevention of atopic dermatitis.

The inventors have shown an inhibitory effect of this combination on the adhesion of Staphylococcus aureus to keratinocytes in culture. The combination of glycerol, vaseline and liquid paraffin therefore allows prevention of the infectious role of Staphylococcus aureus in atopic patients as well as his/her role on sustaining the inflammatory reaction by the production of specific IgEs inhibiting colonization of the skin by Staphylococcus aureus.

Further, the inventors have shown that this combination restores the protective and function barrier role of the skin. For this, the inventors have used a skin model ex vivo of induced skin dehydration. Further, they followed the expression of epidermal markers and serine protease activity.

The object of the present invention is therefore a composition for topic use comprising, as an active ingredient, a glycerol, vaseline and liquid paraffin combination, as an oil-in-water or water-in-oil emulsion for its use for preventing atopic dermatitis.

Preferably, the prevention is of the primary type.

In the sense of the present invention, the terms “prevent”, “prevention” or “preventive treatment” means avoiding the occurrence of a disease, of a disorder or of one or several signs and/or symptoms.

In the sense of the present invention, the term of “primary prevention” or “primary preventive treatment” means preventing the occurrence of atopic diseases (atopic dermatitis and/or allergic bronchial asthma and/or allergic rhinitis commonly called “hay fever”) and/or allergic sensitizations by acting before the first clinical signs of the atopy i.e. from the onset.

In the sense of the present invention, the glycerol, vaseline and liquid paraffin combination as an oil-in-water or water-in-oil emulsion will be called “active combination”.

Advantageously, the glycerol, the vaseline and the liquid paraffin have the criteria described and controlled according to the “European Pharmacopeia”, 6^{th} edition.

Advantageously, the vaseline of the active combination has a drop point comprised between 35 and 70° C., preferably comprised between 51 and 57° C., more preferably of about 54° C. The drop point is measured according to the 2.2.17 method described in “European Pharmacopeia”, 6^{th} edition.

Advantageously, the vaseline of the active combination has a consistency comprised between 175 and 195 1/10 mm, preferably of about 185 1/10 mm (cone penetration at 25° C.)

Advantageously, the vaseline of the active combination has a viscosity comprised between 4 and 5 cSt at 100° C., preferably of about 4.8 cSt at 100° C.

In the composition according to the invention, the active combination is present according to a proportion comprised between 10 and 50% and preferentially between 20 and 30% by weight based on the total weight of the composition; the glycerol concentration is comprised between 5 and 30%, preferentially between 10 and 20% and more preferably is about 15% by weight based on the total weight of the composition, the vaselin concentration is comprised between 3 and 20%, preferentially between 5 and 10% and more preferably is about 8% by weight based on the total weight of the composition and the liquid paraffin concentration is comprised between 0.5 and 5%, preferentially between 1 and 3% and more preferably is about 2% by weight based on the total weight of the composition.

In the aqueous phase, the water is comprised between 30 and 80% by weight based on the total weight of the composition.

Advantageously, the composition according to the invention comprises about 15% of glycerol, about 8% of vaseline and about 2% of liquid paraffin by weight based on the total weight of the composition.

The dermatological composition according to the invention further comprises customary dermatological compatible excipients.

The dermatological composition according to the present invention may be prepared as a water-in-oil (W/O) or oil-in-water (O/W) emulsion, as a multiple emulsion such as for example a water-in-oil-in-water emulsion (W/O/W) or an oil-in-water-in-oil emulsion (O/W/O), or further as a hydrodispersion or lipodispersion, a gel or an aerosol.

The dermatologically compatible excipients may be any excipient among those known to one skilled in the art in order to obtain a composition for topic application as a cream, a lotion, a gel, an ointment, an emulsion, a microemulsion, a spray, etc.

The composition according to the invention may in particular contain additives and formulation aids, such as emulsifiers, thickeners, gelling agents, water fixing agents, spreading agents, stabilizers, dyes, perfumes and preservatives.

Suitable emulsifiers comprise stearic acid, trolamine, PEG-40-stearate.

Preferably, the composition according to the invention has about 5% of emulsifiers by weight based on the total weight of the composition.

Advantageously, the composition according to the invention has between 1 and 5% of stearic acid, preferably about 3% by weight based on the total weight of the composition.

Advantageously, the composition according to the invention has between 0 and 2% of trolamine, preferably about 0.5% by weight based on the total weight of the composition.

Advantageously, the composition according to the invention has between 0 and 2% of PEG-40 stearate, preferably about 0.5% by weight based on the total weight of the composition.

Suitable thickeners comprise glycerol monostearate, PEG.

Preferably, the composition according to the invention has about 5% of thickeners by weight based on the total weight of the composition.

Advantageously, the composition according to the invention has between 2 and 10% of glycerol monostearate, preferably about 5% by weight based on the total weight of the composition.

Suitable preservatives comprise propyl parahydroxybenzoate, chlorocresol.

Preferably, the composition according to the invention has about 0.1% of preservatives by weight based on the total weight of the composition.

Advantageously, the composition according to the invention has between 0.05 and 1% of propyl parahydroxybenzoate, preferably about 0.1% based on the total weight of the composition.

Suitable spreading agents comprise dimethicone, polydimethylcyclosiloxane.

Preferably, the composition according to the invention has about 2% of spreading agents by weight based on the total weight of the composition.

Advantageously, the composition according to the invention has between 0.2 and 2% of dimethicone, preferably about 0.5% by weight based on the total weight of the composition.

Advantageously, the composition according to the invention has between 1 and 3% of polydimethylcyclosiloxane, preferably about 2.5% by weight based on the total weight of the composition.

Suitable water fixing agents comprise polyethylene glycol, preferably polyethylene glycol 600.

Preferably, the composition according to the invention has about 8% of water fixing agents by weight based on the total weight of the composition.

Advantageously, the composition according to the invention has between 2 and 10% of polyethylene glycol, preferably about 5% by weight based on the total weight of the composition.

The water used for the aqueous phase of the emulsion may be distilled water or thermal water having dermato-cosmetic properties.

Advantageously, the composition according to the invention comprises:
about 15% of glycerol,
about 8% of vaseline,
about 2% of liquid paraffin, and as excipients:
about 1 to 5% of stearic acid,
about 2 to 10% of glycerol monostearate,
about 1 to 3% of polydimethylcyclosiloxane,
about 0.2% to 2% of dimethicone,
about 2 to 10% of polyethylene glycol 600,
about 0 to 2% of trolamine,
about 0.05 to 1% of propyl parahydroxybenzoate,
water up to 100%.

The object of the present invention is also the use of a composition according to the invention for making a drug intended for preventing atopic dermatitis.

The present invention is illustrated by the following examples.

### EXAMPLES

### Example 1

### Formulations

### Composition A

15 g of glycerol,
8 g of vaseline,
2 g of liquid paraffin,
0.5 g of trolamine,
and as excipients, stearic acid, glycerol monostearate, polydimethylycyclosiloxane, dimethicone, polyethylene glycol (PEG) 600, propyl parahydroxybenzoate
water up to 100 g.

### Composition A′

15 g of glycerol,
8 g of vaseline,
2 g of liquid paraffin,
1.5 g of stearic acid,
5 g of glycerol monostearate,
1.5 g of polydimethylcyclosiloxane,
0.5 g of dimethicone,
5 g of polyethylene glycol 600,
0.15 g of trolamine,
0.1 g of propylparahydroxylbenzoate
water up to 100 g.

### Composition B

15 g of glycerol,
8 g of vaseline,
2 g of liquid paraffin,
0.5 g of PEG-40 stearate,
and as excipients, stearic acid, glycerol monostearate, polydimethylycyclosiloxane, dimethicone, polyethylene glycol (PEG) 600, chlorocresol,
water up to 100 g.

### Composition B′

15 g of glycerol,
8 g of vaseline,
2 g of liquid paraffin,
3 g of stearic acid,
5 g of glycerol monostearate,
2 g of polydimethylcyclosiloxane,
0.5 g of dimethicone,
0.1 g of trolamine,
3 g of polyethylene glycol 600,
0.5 g of PEG-40-stearate,
0.075 g of chlorocresol,
water up to 100 g.

### Example 2

### Cytotoxicity Test

### Principle

The principle of the test is based on the enzymatic transformation of a tetrazolium salt, “sodium 3,3-[1[(phenylamino)carbonyl]-3-4-tetrazolium bis(4-methoxy-6-nitro)]benzene sulfonic acid hydrate” i.e. XTT, into a colored product, formazan.

XTT is reduced by the mitochondrial dehydrogenase of living cells in the presence of an electron coupling agent, the coenzyme Q, into a yellow/orangey water-soluble compound, formazan, which may be assayed by spectrophotometry at 450 nm.

### Treatment With the Product

96-well microplates are sown with 10⁵ cells/mL, after 24 hours of incubation, the culture medium is removed and the microplates are rinsed with PBS. 100 μL of the different dilutions of the test product are added into each well of the microplates. Controls without any product are made under the same conditions.

The microplates are placed for incubation for 2 hours at 37° C. under 5% CO₂.

### Revealing Cytotoxicity

After incubation, the microplates are washed twice with PBS. Next, 100 μL of a XTT (1 mg/mL)/coenzyme Q (0.2 mg/mL) mixture are added in each well of the 96-well plate.

After 3 hours of incubation, 100 μL of a 10% SDS solution are added into each well.
Immediate reading of the optical density at 450 nm is carried out with the spectrophotometer POLARstar (BMG, France).

### EXPRESSION OF THE RESULTS

The measured optical density is proportional to the viable cell population.

With this test, it is therefore possible for us to analyze from an optical density corresponding to the control, cell cytotoxicity when the optical density is less than that of the control batch:

% viability=(Treated OD−Control OD)/Control OD×10

The product is considered cytotoxic if the viability percent is ≦30%.

The intra-manipulation tests are carried out 8 times.

### RESULTS

Depending on the degree of non-cytotoxicity of the products, the concentrations selected for the adhesion test are the following:
Composition A: 6%, 3%, 1.5%, 0.8% and 0.4%
Atopiclair®: 0.8%, 0.4%, 0.2%, 0.1% and 0.05%
Physiogel®: 6%, 3%, 1.5%, 0.8% and 0.4%
Composition B: 3%, 1.5%, 0.8%, 0.4% and 0.2%

### Example 3

### Adhesion Test

### Principle

Labelling the bacteria with tritiated adenine (sigma) and determining the proportion of bacteria adhered to the surface of the keratinocytes by evaluating the radioactivity level.

### Labelling the Bacteria With Tritiated Adenine

Labelling of the bacteria is carried out in the presence of 30 μCi of tritiated adenine in an adequate culture medium. After incubation for 18 hours at 37° C., the microorganisms are washed three times in PBS in order to remove non-incorporated radioactivity.

The suspensions are adjusted 2×10⁸ microorganisms/mL in the sustaining medium.

### Adhesion Inhibiting Effect

500 μL of labelled bacteria in the presence of each test dilution of the product are deposited at the surface of the cell layer.

After 2 hours incubation (37° C., 5% CO₂), 3 washings with PBS are carried out.

### Lysis of the Cells

The bacteria adhered to the keratinocytes are lyzed with 500 μL of a 0.5 N sodium hydroxide solution with 0.1% of sodium dodecyl sulfate for 18 hours at 37° C.

The lysate of each well is sampled and placed in counting tubes. 2 mL of liquid scintillator are added in each tube.

### EXPRESSION OF THE RESULTS

Reading is carried out with a β counter which expresses the radioactivity level in cpm (counts per minute).

The adhesion inhibition percentage is calculated according to the formula:

inhibition %=((test cpm−control cpm)/control cpm)×100

The adhesion inhibition percentage is significant if its value is ≦−30%.

The tests are conducted in quadruplicate.

### CONCLUSION

Composition A significantly inhibits adhesion of S. aureus to keratinocytes after 2 hours of incubation at 6% and 3%.
The Physiogel product significantly inhibits adhesion of S. aureus to keratinocytes after 2 hours of incubation at 6% but not at 3%.
The Atopiclair product does not have any effect on the adhesion of S. aureus to the keratinocytes.
In the presence of the composition B, the adhesion of S. aureus to the keratinocytes is abnormally increased.

### Example 4

### Analysis of the Regulation of Induced Skin Dehydration

We evaluate here the moisturizing activity of composition A and the subsequent improvement in the barrier function of the skin by using a skin model ex vivo of induced skin dehydration.

We observe the expression of the differential molecular epidermal markers by quantitative PCR and immuno-histochemistry.

We also track the activity of serine protease enzymes by zymography in situ and the degradation of corneodesmosomal proteins by Western blotting.

The functionality of the skin barrier is analyzed by using fluorescent probes.

### Equipment and Methods

### 1. Tissue Models

### 1. Preparation of Skin Explants

The laboratory recovers skin samples from operatory wastes of plastic surgery (mammary reductions). The use of these samples enters the scope of “the declaration of an activity for preserving and preparing elements of the human body for the needs of a research program of the Pierre Fabre group” made to the French Ministry of Higher Education and Research.

These samples are washed in 10 PBS baths and then die-cut into disks with a diameter of 2 cm. The skin explants are spread on a grid in a Petri dish and a ring with a diameter of 1 cm is sealed on the skin in order to delimit the treatment area.

### 2. Kinetics of the Models

For the induced dehydration model, the skin is dried for 2 hours under a cell culture hood in a dish without any lid and then put into the oven for topic treatment with or without any active combination for 2 hours. The negative control of the dehydration stress undergoes the same kinetics in a closed Petri dish.

### 3. Samples for Analysis

After the treatment, 2 biopsies with a diameter of 6 mm are sampled for analyzing the expression of RNAs and a biopsy with a diameter of 4 mm included in a Tissue Tek® (Sakura Finetek) resin bloc for histology. For analyzing the protein, the skin is exposed to a thermal shock in a water bath at 60° C. for 5 minutes and then at 4° C. for 2 minutes in order to separate the epidermis from the dermis.

The biopsies and epidermises are frozen in liquid nitrogen and stored at −80° C. before being analyzed.

### II. Analysis of the Transcriptome by Quantitative PCR

The skin biopsies are milled in a mortar cooled beforehand with liquid nitrogen and the RNAs are extracted by means of an RNeasy® kit (QIAGEN) according to the recommendations of the supplier. The RNA is then assayed with a Bioanalyzer 2100® (Agilent Technologies) on RNA 6000 Nano LabChip® chips. cDNA is obtained from 1 μg of RNA by an enzymatic retrotranscription reaction carried out with an Access RT-PCR Core Reagents® kit (Promega) with oligo dT primers. The gene expression levels are analyzed by quantitative PCR on a fluorescence thermocycler IClycler iQ® (Biorad) with PCR iQ™SYBR® Green Super Mix kits (Biorad) according to a 40 cycle procedure comprising denaturation at 95° C. (15 s) and elongation at 60° C. (1 mm). The accumulation of the PCR product, proportional to the fluorescence emission (SYBR®Green intercalant) is viewed cycle after cycle by means of the iCycler software.

The iClycler version 3.1 analysis software delivers raw values of C_{T} (Cycle Threshold): cycle from which begins cDNA amplification. The expression of several reference genes is analyzed in parallel by means of the program Genorm version 3.4 which allows selection of the most stable reference gene from one sample to the other. This gene is then used as a reference for normalization of the results by calculating ΔC_{T}=gene of interest C_{T—}reference gene C_{T}.

The induction factor (IF) is then calculated for each treatment relatively to the corresponding control condition. IF=2^{-ΔΔCT} wherein ΔΔCT=treated ΔCT-control ΔCT. The expression of the mRNAs is evaluated in duplicate for five experiments from 5 different individuals. When the induction factor relatively to the control is greater than 2, it is considered that the expression of the gene is induced and when it is less than 0.5, it is considered that the expression is repressed. The effect of the active ingredient on the response to the stress caused in the model is evaluated by the inhibition percentage calculated with the following formula:

Stress response inhibition %=100*[(stressed IF-stress-free control IF)−(treated IF-stress-free control IF)]/[(stressed IF-stress-free IF)]

As compared with the study model, the “stress-free control” condition corresponds to a non-dried control; the “stressed” condition corresponds to a skin biopsy which has been dried for 2 hours and which has spent 2 additional hours under the control conditions (i.e. without any topic treatment); finally the “treated” condition is the skin which has undergone 2 hours of drying followed by 2 hours of topic treatment with an emollient.

### III. Analysis of the Protein Expression by Western Blotting

The treated epidermises are milled in a mortar cooled with liquid nitrogen and the proteins are extracted in a lysis buffer ROPA (Tris HCl pH8 50 mM; NaCl 150 mM; Triton×100 IX; Na+desoxycholate 1%; SDS 0.1%; EDTA 5 mM; DTT 100 mM; a cocktail of protease inhibitors (reference P8340, SIGMA).

The proteins are then assayed by the DC-DC Protein Assay method (Biorad) and analyzed by Western blotting. For each condition, 25 to 40 μg of total proteins are deposited on Tris-Glycine gels with 7.5% polyacrylamide. The protein mixture is separated by electrophoresis with the Mini Protean II system (Biorad) and the proteins are transferred on a PVDF membrane (Hybond-P, Amersham). The protein of interest is revealed by a specific antibody and an ECL+ kit (Amersham). The amount of proteins and the proportion of degraded form are calculated by means of the software Image Master TotalLab version 1.11 (Amersham) after normalization relatively to β-actin (reference protein).

### IV. Histological Techniques

The skin biopsies are severed with the Cryotome (Leica CM 3050s) into cuts with a thickness of 5 μm and deposited on observation slides (Starfrost®).

### 1. Immunohistochemistry

The cryocuts are fixed for 10 minutes with acetone at 20° C. and then rehydrated with PBS before being analyzed by immunochemical labelling. After fixing and rehydration, the skin cuts are saturated with a 3% BSA solution and incubated for 1 hour with the primary antibody directed against the protein of interest. In a second phase, they are incubated for 1 hour with the secondary antibody coupled with a Alexa-488 or Alexa-555 fluorochrome and finally mounted in Mowiol containing DAPI for labelling the nuclei.

### 2. Zymography In Situ

After fixing for 10 minutes in acetone at −20° C., the cuts are rinsed in a washing solution (1% Tween 20 in water) and incubated for 2 hours at 37° C. with a solution containing the specific substrate of the enzymes of interest coupled with a fluorophore (ancillary). When the enzyme is activated, the fluorophore is cleaved, releasing a fluorescent signal which may be observed under the microscope. The labelled slides are then observed under a microscope with epifluorescence (Nikon Eclipse 50i) or under an inverted confocal microscope Zeiss Axiovert 100.

### 3. Fluorescent Probe

After the dehydration treatment, the skin explants are further incubated for one hour in the oven at 37° C. with a fluorescent probe Lucifer Yellow carboxy-hydrazide dilithium salt (Invitrogen) at 1 mM in HBSS buffer. The skin is then rinsed in a HBSS bath for 1 minute and then biopsies with a diameter of 4 mm are sampled and included in the Tissue TekR® resin (Sakura Finetek) (Matsuki et al., 1998). The skin is then cut, the nuclei are marked with DAPI and the slides are observed under a fluorescence microscope with a wavelength of 450 nm as described above.

### RESULTS

### 1. Barrier Function Failure Model by Induced Drying

### 1. Measurement of Skin Permeability With a Fluorescent Probe

The first analysis consisted of studying a fundamental functional parameter in the skin barrier function: the permeability of the upper layers of the epidermis. Incubation of the skin with a fluorescent probe (Lucifer Yellow) after the drying experiment allowed characterization of the modulation of skin permeability. Under control conditions, the labelling is very low and superficial, the probe does not penetrate very much through the corneum stratum and is removed during rinsing. After two hours of drying, the labelling is observable in the deeper strata of the corneum stratum. Drying makes the skin more permeable, its barrier function is deteriorated. The topic treatment by composition A following two hours of drying restores the impermeability of the SC relatively to the probe, the labelling is again low and superficial, like under the control conditions. It may then be concluded that the moisturizing treatment has a repairing effect on the dried skin and on the skin barrier function which is observable on the tissue model.

### 2. Effects on the Regulation of the Transcriptome and of the Proteome

The expression of different genes potentially involved in homeostasia of the epidermal barrier function was measured by quantitative PCR under different stress or treatment conditions of the drying model. Analysis by immunohistochemistry gave the possibility of showing the reorganization of the expression of certain proteins in terms of localization, for example close junctions. The degradation of corneodesmosomal proteins was analyzed by Western blotting.

The targets studied by means of these different approaches were grouped together according to their physiological role (see Table 1). The goal of the study is to observe a observable stress response and a correction of the stress effect by topic application of composition A.

With the accomplished work it was also possible to show the different regulation levels of certain targets. Thus, we were able to notice that the enzymes of desquamation were not regulated at a transcriptional level but more particularly at their activity level (cf. results 3).

TABLE 1
Summary of the targets and of the
pharmacological response studied in the dryness model

| | Response to | Stress response inhibition by composition |
|---|---|---|
| Target | stress | A |
| Skin | Y | Y |
| permeability | | |
| Desmoglein 1 | Y | Y |
| Cdsn | X | X |
| Desmocollin | Y | Y |
| Plakoglobin | Y | X |
| KLK5 | Y | X |
| KLK7 | X | X |
| KLK8 | X | X |
| Serine | Y | Y |
| proteases | | |
| Cathepsin D | X | X |
| ABCA12 | Y | Y |
| ABCG1 | X | X |
| B-GC | X | X |
| DES 2 | X | X |
| Filaggrin | Y | Y |
| Involucrin | X | X |
| TG1 | X | X |
| TG3 | X | X |
| Caspase 14 | X | X |
| ElYx-3 | Y | X |
| 12R-LYX | X | X |
| HAS 2 | Y | X |
| CD44 | X | X |
| AQP3 | Y | Y |
| NHE1 | Y | Y |
| IL-la | Y | Y |
| Occludin | Y | Y |
| Claudin 1 | Y | Y |
| Claudin 4 | Y | Y |
| ZO-1 | X | X |
| E-Cadherin | X | X |
| β-Catenin | X | X |

Y = The target reacts in the model; Y = Yes
X = the target does not react in the model N = No

### 3. Measurement of Enzymatic Activity Related to Desquamation

The activity of serine protease was evaluated by zymography in situ on the dehydration model and observed under a confocal microscope under control conditions after two hours of drying and after two hours of drying followed by incubation for two hours with the composition A. The labelling is more intense under control conditions, it corresponds to normal strong activity. This labelling decreases and becomes irregular along the corneum stratum after two hours of drying while its intensity is increased and the localization of the activity is reorganized after two hours of incubation with composition A. The drying has the effect of decreasing and perturbing enzymatic activity. These results are consistent with the reduction in the degradation of corneodesmosomal proteins which we observe with the drying and confirms the effect of drying on the reduction in desquamation observed on the developed model. Composition A is capable of restoring the enzymatic activity of the dehydrated skin which confirms the effect of this composition for a return of homeostasia of the desquamation.

These results show that the composition A allows restoration of the expression level of the molecular targets, the expression of which is increased by the induced stress of skin dehydration. With the composition A it is also possible to restore serine protease activity. Further, topic application of composition A allows suppression of stress-induced inflammation.

The whole of these results suggests that the composition A as a topic application allows restoration of the barrier function of the skin, limitation or even prevention of colonization by the golden staphylococcus and thus is a “primary preventive treatment” preventing the occurrence of atopic diseases (atopic dermatitis and/or allergic bronchial asthma and/or allergic rhinitis commonly called “hay fever”) and/or allergic sensitizations.

1.-12. (canceled)

13. A method for the prevention of atopic dermatitis comprising the topical administration of a composition comprising, as an active ingredient, a combination of glycerol, vaseline and liquid paraffin as an oil-in-water or water-in-oil emulsion to a patient in need thereof.

14. The method according to claim 13 for the primary prevention of atopic dermatitis.

15. The method according to claim 13, wherein the vaseline has a drop point comprised between 35 and 70° C.

16. The method according to claim 15, wherein the vaseline has a drop point comprised between 51 and 57° C., notably 54° C.

17. The method according to claim 13 wherein the vaseline has a consistency comprised between 175 and 195 1/10 mm (cone penetration at 25° C.).

18. The method according to claim 17, wherein the vaseline has a consistency of about 185 1/10 mm (cone penetration at 25° C.).

19. The method according to claim 13, wherein the vaseline has a viscosity comprised between 4 and 5 cSt at 100° C.

20. The method according to claim 19, wherein the vaseline has a viscosity of about 4.8 cSt at 100° C.

21. The method according to claim 13 comprising about 15% of glycerol, about 8% of vaseline and about 2% of liquid paraffin.

22. The method according to claim 13, comprising one or more excipients selected from the group consisting of stearic acid, glycerol monostearate, polydimethylcyclosiloxane, dimethicone, polyethylene glycol 600, trolamine, propyl parahydroxybenzoate, distilled water.
